(19) United States
(12) Patent Application Publication
(10) Pub. No.: US 2011/0184208 A1
(43) Pub. Date: Jul. 28, 2011

(54) CATALYTIC OXIDATION REACTIONS IN SUPERCRITICAL OR NEAR-SUPERCRITICAL WATER FOR THE PRODUCTION OF AN AROMATIC CARBOXYLIC ACID

(76) Inventors: Joan Fraga-Dubreuil, Cleveland (GB); Samuel Duncan Housley, North Yorkshire (GB); Walter Partenheimer, Portland, OR (US)

(21) Appl. No.: 12/989,960

(22) PCT Filed: Apr. 29, 2009
(86) PCT No.: PCT/US09/42096

§ 371 Date: Dec. 3, 2010

(30) Foreign Application Priority Data
Apr. 30, 2008 (GB) 0807904.8

(51) Int. Cl. C07C 63/04; C07C 51/16

(52) U.S. Cl. 562/418; 562/408; 562/493

(57) ABSTRACT

An oxidation process for the production of an aromatic carboxylic acid, said process comprising contacting in the presence of a catalyst, within a continuous flow reactor, one or more precursor(s) of the aromatic carboxylic acid with an oxidant, such contact being effected with said precursor(s) and the oxidant in an aqueous solvent comprising water under supercritical conditions or near supercritical conditions, wherein said catalyst comprises copper.

### CROSS-REFERENCE TO RELATED APPLICATION

This application claims benefit of priority from Great Britain Application No. 0807904.8 filed Apr. 30, 2008.

### FIELD OF THE INVENTION

This invention relates to synthetic catalytic oxidation processes in supercritical or near-supercritical water, particularly the oxidation of alkyl-substituted aromatic hydrocarbons to the corresponding aromatic carboxylic acid, particularly terephthalic acid, isophthalic acid, trimellitic acid and naphthalene dicarboxylic acid.

### BACKGROUND OF THE INVENTION

The dielectric constant of water decreases dramatically from a room temperature value of around 80 C²/Nm² to a value of 5 C²/Nm² as it approaches its critical point (374° C. and 220.9 bara), allowing it to solubilise organic molecules. As a consequence, water then behaves like an organic solvent to the extent that hydrocarbons, e.g. toluene, are completely miscible with the water under supercritical conditions or near supercritical conditions. Terephthalic acid, for instance, is virtually insoluble in water below about 200° C. Dioxygen is also highly soluble in sub- and super-critical water.

Holliday R. L. et al (J. Supercritical Fluids 12, 1998, 255-260) describe a batch process carried out in sealed autoclaves for the synthesis of, inter alia, aromatic carboxylic acids from alkyl aromatics in a reaction medium of sub-critical water using molecular oxygen as the oxidant. A number of different catalyst systems were investigated in Holliday's studies, which showed that the bromide salt of either Mn(II) or Co(II) must be utilised in order to facilitate the complete oxidation of the alkyl aromatic substrate to the corresponding aromatic carboxylic acid. Holliday reported that Fe(II) and Ni(II) salts were disadvantageous since they produced large amounts of carbonaceous material. Copper bromide was found to the most efficient catalyst for the oxidation of toluene to benzaldehyde but otherwise produced severe charring and coupling reactions, and therefore disadvantageous for the production of aromatic carboxylic acids.

Copper has previously been reported as having poor or no catalytic activity for the oxidation of p-xylene or other alkylaromatics to the corresponding carboxylic acid in conventional conditions and solvents, either alone or as a co-catalyst (see, for instance, W. Partenheimer, J. Mol. Catal., 67, (1991), 35-46; Alper et al., J. Mol. Catalysis, vol. 61, p. 51-54, 1990; M. Hronec and A. Bucinska, Oxid. Commun. 10(3)(1987) 193; Okada and Kamiya Bull. Chem. Soc. Japan, Vol. 54(9), 2724-7, 1981 and Bull. Chem. Soc. Japan, Vol. 52, 3321, 1979; V. N. Aleksandrov, Kinetika i Kataliz, 19(4), 1057-1060, 1978; and U.S. Pat. No. 3,299,125). Indeed, the addition of copper to a mixed cobalt/manganese/bromide catalyst for alkylaromatic oxidation reactions in acetic acid solvent has been reported to inhibit the oxidation reactions (G. H. Jones, J. Chem. Res., Synopses (1982), (8), 207; and Y. Kamiya et al., Bull. Chem. Soc. Japan. Vol 39(10), 2211-15, 1966). GB-644667 describes the oxidation in acetic acid of p-tolualdehyde to faun primarily p-toluic acid but with a minor amount of terephthalic acid also formed using cobalt and copper acetate as a catalyst in the absence of bromide, but very long residence times were required. Borovkova et al. (Neftekhimiya, 16, 235 (1976)) describe the oxidation of pseudocumene (1,2,4-trimethylbenzene) in a solventless system and in the absence of bromide using cobalt and manganese acetates and iron and copper dimethylbenzoates as catalysts. The authors report low catalytic activity for the copper and iron salts, whereas the cobalt and manganese salts ensured rapid conversion of intermediate products to acids. The addition of copper to a cobalt acetate catalyst at low Cu/Co ratios (Cu/Co<0.1) showed weak synergy in the oxidation of pseudocumene, which is manifested in an increased oxidation rate and an increased acid yield, although this was observed only in respect of an intermediate product, rather than in the formation of trimellitic acid. However, increasing the concentration of the metal additive to Cu/Co ratios greater than 0.1 deactivates the cobalt catalyst, leading to a reduction in the oxidation rate and then complete inhibition of the oxidation reaction. JP-58/023643-A discloses the preparation of aromatic dicarboxylic acids by the oxidation of xylene in an aqueous solvent containing a bromine compound and a water-soluble copper salt under conditions of relatively low pressure and temperature, and teaches that xylene combustion becomes severe at temperatures above 260° C., with reduced product yield. DE-10/2006/016302-A discloses the oxidation of an alkylbenzol in a water-containing solvent and a heterogeneous (i.e. solid) catalyst which is an oxide of Ce, Fe, Co, Mn, V, Ti, Zr and/or Cu, using a temperature less than 350° C. and a pressure in the range of 20 to 80 bar, preferably wherein the temperature is from 280 to 320° C. and the pressure is from 25 to 35 bar wherein water is in the vapour phase, and discloses a reduction in catalysis performance at higher pressures.

The use of supercritical water as a medium for the production of aromatic carboxylic acids in a continuous flow reactor was first disclosed in WO-02/06201-A. The process taught therein comprised contacting in the presence of an oxidation catalyst, within a continuous flow reactor, one or more precursors of the aromatic carboxylic acid with an oxidant, such contact being effected with said precursor(s) and the oxidant in an aqueous solvent comprising water under supercritical conditions or near supercritical conditions close to the supercritical point such that said one or more precursors, oxidant and aqueous solvent constitute a substantially single homogeneous phase in the reaction zone. In the process described in WO-02/06201-A, the contact of at least part of the precursor with the oxidant is contemporaneous with the contact of the catalyst with at least part of the oxidant. The oxidation catalyst disclosed in WO-02/06201-A comprises one or more heavy metal compounds, e.g. cobalt and/or manganese compounds such as bromides, bromoalkanoates or alkanoates (usually C1-C4 alkanoates such as acetates). Compounds of other heavy metals, such as vanadium, chromium, iron, molybdenum, a lanthanide such as cerium, zirconium, hafnium, and/or nickel are also envisaged in WO-02/06201-A, and the oxidation catalyst may alternatively or additionally include one or more noble metals or compounds thereof, e.g. platinum and/or palladium or compounds thereof. In the continuous process of WO-02/06201-A the reaction kinetics are further enhanced by the high temperatures prevailing when the water solvent is under supercritical or near supercritical conditions. The combination of high temperature, high concentration and homogeneity mean that the reaction to convert the precursor(s) to aromatic carboxylic acid can take place extremely rapidly compared with the residence times employed in the production of aromatic carboxylic acids such as terephthalic acid by conventional techniques using a crystallising three phase oxidation reactor. Under these conditions, the intermediate aldehyde (e.g. 4-carboxybenzaldehyde (4-CBA) in the case of terephthalic acid) is readily oxidised to the desired aromatic carboxylic acid which is soluble in the supercritical or near supercritical fluid thereby allowing a significant reduction in contamination of the recovered aromatic carboxylic acid product with the aldehyde intermediate. The process conditions of WO-02/06201-A substantially reduce or avoid autocatalytic destructive reaction between the precursor and the oxidant and consumption of the catalyst. The continuous process involves short residence times and exhibits high yield and good selectivity of product formation.

Dunn and Savage (in Environ. Sci. Technol. 2005, 39, 5427-5) studied the effect of oxygen concentration and catalyst concentration and identity in the partial oxidation of p-xylene to terephthalic acid using high-temperature liquid water as solvent in a batch process. That study reinforced the preference for MnBr₂ as the catalyst in this oxidation reaction, relative to CoBr₂, ZrBr₄ and Mn(OAc)₂.

While the preferred catalyst in the supercritical oxidation for the production of aromatic carboxylic acids comprises manganese salts (particularly MnBr₂), it has been observed that manganese salts are oxidised irreversibly to manganese oxide(s) (including MnO₂, Mn₂O₃ and MnO(OH)₂) during the strong oxidative conditions of the reaction. The manganese oxide(s) forms an insoluble precipitate which adheres to internal walls following the initial contact between the catalyst and the oxidant (typically molecular oxygen), resulting in the progressive fouling of the reactor and/or blockages in the pressure let-down equipment. This precipitation of manganese oxide(s) reduces or prevents the opportunity to recycle catalyst for effective operation of the process, and this loss of catalyst is economically undesirable. In addition, the precipitation reduces or prevents flow in a tubular reactor, and the channels in the apparatus need to be cleaned or unblocked in order to continue operation of the reactor, which is uneconomic and inefficient. The specific mixing configuration described in WO-02/06201-A minimises catalyst oxidation compared to other configurations, thereby minimising reactor fouling.

It remains desirable to make improvements in the oxidation reaction for the production of aromatic carboxylic acids, in particular to improve the yield of, and/or the selectivity for, the target compounds. Another important consideration is minimising the “burn” of the reaction. As used herein, the “burn” of the reaction is defined as the non-selective oxidation and/or degradation of the precursor(s), oxidation intermediate(s) and/or target end-product(s) which can ultimately proceed through to the carbon oxide(s), as opposed to the selective oxidation of the precursor(s) to the target compound(s). Burn is quantified in one embodiment by the proportion of carbon oxide(s) generated by the reaction. In addition, it remains desirable to avoid the fouling of the reactor as described above in order to retain the essential operability of the oxidation process, particularly while maintaining or improving yield and/or selectivity and/or burn.

### SUMMARY OF THE INVENTION

It is an object of this invention to reduce or avoid one or more of the above-mentioned problems. In particular, it is an object of this invention to provide an alternative or improved continuous process for the production of an aromatic carboxylic acid via catalytic oxidation of a precursor, particularly such a process having one or more of (i) good selectivity for the aromatic carboxylic acid, and/or (ii) high yield of the aromatic carboxylic acid; and/or (iii) low burn. It is a further object of this invention to provide an alternative or improved continuous process for the production of an aromatic carboxylic acid via catalytic oxidation of a precursor, particularly such a process wherein the catalyst system allows a reduction in the amount of catalyst required, relative to MnBr₂, without detriment to selectivity and/or yield of the aromatic carboxylic acid and/or without increasing burn. It is a further object of this invention to avoid the fouling of the reactor in order to retain the essential operability of the oxidation process, particularly while maintaining or improving yield and/or selectivity and/or burn. It is a further object to provide an alternative or improved catalyst system for the supercritical (or near-supercritical) water synthetic oxidation process for the production of aromatic carboxylic acids.

According to the present invention there is provided an oxidation process for the production of an aromatic carboxylic acid, said process comprising contacting in the presence of a catalyst, within a continuous flow reactor, one or more precursor(s) of the aromatic carboxylic acid with an oxidant, such contact being effected with said precursor(s) and the oxidant in an aqueous solvent comprising water under supercritical conditions or near supercritical conditions, typically such that said one or more precursor(s), oxidant and aqueous solvent constitute a single homogeneous phase in the reaction zone, wherein said catalyst comprises copper.

When compared with WO-02/06201-A, the catalyst system of the process according to the present invention provides an unexpected improvement in selectivity and/or yield of the target compound(s), and/or exhibits a reduction in burn. In addition, the copper-containing catalysts described herein advantageously exhibits a reduced tendency for the reactor to be fouled as a result of catalyst precipitation.

### BRIEF DESCRIPTION OF THE DRAWINGS

FIG. 1 is a schematic flowsheet illustrating the basic arrangement described for Embodiment I below.

FIGS. 2A and 2B are schematic flowsheets illustrating the basic arrangement described for Embodiment II below. In FIG. 2B, the oxidant is introduced in a progressive manner along the reaction zone at multiple injection points.

FIG. 3 is a schematic flowsheet illustrating an arrangement (such as Embodiment III below) where contact of the precursor and oxidant is non-contemporaneous with contact of catalyst and oxidant.

FIG. 4 is a schematic flowsheet illustrating in more detail an arrangement wherein the precursor is added to a premixed stream of oxygen and water (i.e. an arrangement according to the process illustrated in FIG. 1);

FIGS. 5A, 5B, 5C, 5D and 6 illustrate various premixer configurations that can be employed to effect mixing of at least one of the reactants with the aqueous solvent;

FIG. 7 is a schematic view illustrating multiple stage injection of oxidant;

FIGS. 8 and 9 are schematic flowsheets illustrating mother liquor recycle and heat removal from a reactor for use in oxidising a terephthalic acid precursor in supercritical or near supercritical water, substantially pure oxygen being used as the oxidant in the embodiment of FIG. 8 and air being the oxidant in the embodiment of FIG. 9.

FIG. 10 is a detailed illustration of the apparatus used for the laboratory-scale experiments.

### DETAILED DESCRIPTION OF THE INVENTION

By the term “synthetic oxidation reaction” we mean the production of one or more target compound(s) from one or more oxidisable precursor(s) thereof by partial oxidation of said precursor(s). By the term “partial oxidation” we mean an oxidation reaction which consists of a degree of oxidation (or uptake of oxygen) less than that required for total oxidation of said precursor(s) to carbon oxides; such reactions are associated with controlled oxidant/precursor stoichiometry, selective reaction for the synthesis of a small number of compounds in high yield, and retention of chemical structure in the aromatic group of the precursor. By the term “total oxidation” we mean oxidation of a compound to carbon oxides (typically carbon dioxide), i.e. destructive oxidation.

The pressure and temperature of the process are selected to secure supercritical or near-supercritical conditions. As used herein, the term “near-supercritical conditions” means that the solvent is at a temperature which is not less than 100° C. below the critical temperature of water at 220.9 bara. In one embodiment, the solvent is at a temperature is not less than 80° C. below, and in a further embodiment not less than 70° C. below, and in a further embodiment not less than 50° C. below, and in a further embodiment not less than 35° C. below, and in a further embodiment not less than 20° C. below the critical temperature of water at 220.9 bara. Thus, operating temperatures are typically in the range of from about 280 to about 480° C., more preferably from about 280 to about 380° C., typically from about 300 to about 370° C., particularly from about 300 to about 340° C. Operating pressures are preferably at least about 64 bara, preferably at least about 71 bara, preferably at least about 81 bara, and more preferably at least about 86 bara, and preferably no more than about 350 bara, preferably no more than about 300 bara, and preferably no more than about 250 bara. In a preferred embodiment, the operating pressures are in the range from about 64 to about 350 bara, preferably from about 81 to about 350 bara, more preferably from about 86 to about 350 bara, more preferably from about 180 to about 250 bara, and in one embodiment from about 200 to about 230 bara. In a preferred embodiment, the temperature is at least 280° C., and the pressure at least 64 bara. In the embodiments of the invention relating to near-supercritical conditions, temperature and pressure are preferably selected such that the reaction conditions fall within the liquid phase region of the phase diagram of water (pressure (y-axis) plotted against temperature (x-axis)).

In a preferred embodiment, the term “near-supercritical conditions” means that the reactants and the solvent constitute a single homogeneous phase. By the term “single homogeneous phase” as used herein, we mean that at least 80%, typically at least 90%, typically at least 95%, more typically at least 98%, and most typically effectively all, by weight, of each of the precursor, oxidant, aqueous solvent, catalyst and reaction product(s) are in the same single homogeneous phase in the reaction zone.

By the term “aromatic carboxylic acid” as used herein, we mean an aromatic compound in which a carboxylic acid group (—CO₂H) is attached directly to an aromatic group (Ar). The aromatic carboxylic acid may contain one or more carboxylic acid groups attached directly to an aromatic group, and the present invention is particularly directed to aromatic carboxylic acids which contain at least 2, and particularly only 2, carboxylic acid groups (CO₂H) attached directly to an aromatic group. One or more substituent group(s) other than hydrogen and carboxylic acid group(s) may also be attached directly to the aromatic group (Ar), such as alkoxy groups (particularly C₁₋₄ alkoxy groups, and particularly methyl), but typically the substituent groups attached directly to the aromatic group (Ar) are selected from the group consisting of hydrogen and carboxylic acid group(s). The aromatic group (Ar) may comprise a single aromatic ring or may comprise two or more aromatic rings, for instance two or more fused aromatic rings, the or each ring typically having 5, 6, 7 or 8 ring atoms, more typically 6 ring atoms. Typically, the aromatic group is mono-cyclic. The aromatic group may be a carbocyclic aromatic group or it may comprise one or more heterocyclic aromatic rings (for instance those containing 1, 2 or 3 heteroatoms (typically only 1 heteroatom) selected from N, O and S, typically N). In one embodiment, the aromatic group is phenyl. In an alternative embodiment, the aromatic group is pyridyl. Typical aromatic carboxylic acids which may be synthesised using the present invention include terephthalic acid, isophthalic acid, phthalic acid, trimellitic acid, naphthalene dicarboxylic acid, nicotinic acid and anisic acid. The present invention is particularly directed to the production of terephthalic acid, isophthalic acid, phthalic acid and naphthalene dicarboxylic acid, and particularly terephthalic acid.

By the term “precursor of an aromatic carboxylic acid” as used herein we mean an aromatic compound which is oxidisable to the target aromatic carboxylic acid with an oxidant under supercritical conditions or near-supercritical conditions. The precursor is selected from aromatic compounds having at least one substituent which is attached to the aromatic group (Ar; as defined hereinabove) and which is oxidisable to a carboxylic acid moiety. Suitable substituents are typically selected from alkyl, alcohol, alkoxyalkyl and aldehyde groups, particularly C₁₋₄ alkyl, C₁₋₄ alcohol, (C₁₋₄ alkoxy)C₁₋₄ alkyl and C₁₋₄ aldehyde groups, and preferably alkyl groups (preferably C₁₋₄ alkyl groups, preferably methyl). Where two or more substituent groups are present, these may be the same or different, but are preferably the same. For instance, a precursor of terephthalic acid may be selected from para-xylene, 4-tolualdehyde and 4-toluic acid, para-xylene being preferred. A precursor of nicotinic acid is, for instance, 3-methylpyridine. In situations where the precursor exhibits two or more substituent groups, it is preferred that each substituent group is oxidised to a carboxylic acid group in the oxidation process. In one embodiment, however, a precursor may also exhibit one or more substituent group(s) attached directly to the aromatic group (Ar) which is not oxidisable to a carboxylic acid group and which may be more resistant to oxidation relative to the substituent groups mentioned above, and such groups can include for instance alkoxy groups (particularly a C₁₋₄ alkoxy group, and particularly methoxy).

The reactor suitable for the performance of the present invention is a continuous flow reactor. By “continuous flow reactor” as used herein we mean a reactor in which reactants are introduced and mixed and products withdrawn simultaneously in a continuous manner, as opposed to a batch-type reactor. For example, the reactor may be a tubular flow reactor (with either turbulent or laminar flow) or a continuous stirred-tank reactor (CSTR) although the various aspects of the invention defined herein are not limited to these particular types of continuous flow reactor. By carrying out the process in a continuous flow reactor, the residence time for the reaction can be made compatible with the attainment of conversion of the precursor(s) to the desired aromatic carboxylic acid without significant production of degradation products. The residence time of the reaction medium within the reaction zone is generally no more than 10 minutes, preferably no more than 8 minutes, preferably no more than 6 minutes, preferably no more than 5 minutes, preferably no more than 3 minutes, preferably no more than 2 minutes, preferably no more than 1 minute, and in one embodiment no more than about 30 seconds, for instance about 0.1 to 20 seconds.

The residence time may be controlled so that the precursor is converted to the aromatic carboxylic acid with high efficiency such that the aromatic carboxylic acid precipitated from the reaction medium following completion of the reaction contains no more than about 5000 ppm, preferably no more than about 3000 ppm, more preferably no more than about 1500 ppm, more preferably no more than about 1000 ppm and most preferably no more than about 500 ppm aldehyde produced as an intermediate in the course of the reaction (e.g. 4-CBA in the case of terephthalic acid production). Typically, there will be at least some aldehyde present after the reaction, and usually at least 5 ppm.

The oxidant in the process of the invention is preferably molecular oxygen, e.g. air or oxygen enriched air, but preferably comprises gas containing oxygen as the major constituent thereof, more preferably pure oxygen, or oxygen dissolved in liquid. The use of air is not favoured, although not excluded from the scope of the invention, since large compression costs would arise and off-gas handling equipment would need to cope with large amounts of off-gas owing to the high nitrogen content of air. Pure oxygen or oxygen-enriched gas on the other hand permits use of a smaller compressor and smaller off-gas treatment equipment. The use of dioxygen as the oxidant in the process of the invention is particularly advantageous since it is highly soluble in water under supercritical or near supercritical conditions.

Instead of molecular oxygen, the oxidant may comprise atomic oxygen derived from a compound, e.g. a liquid phase compound at room temperature, containing one or more oxygen atoms per molecule. One such compound for example is hydrogen peroxide, which acts as a source of oxygen by reaction or decomposition.

In the oxidation reaction according to the present invention, the copper-containing oxidation catalyst is homogeneous and soluble in the reaction medium which also comprises solvent and precursor(s). The copper-containing catalyst optionally comprises one or more other metals, particularly a transition metal such as manganese, cobalt, zirconium, hafnium, vanadium, chromium, molybdenum, iron, nickel or cerium, as well as non-transition metals. In one embodiment, the copper-containing catalyst system comprises one or more additional metals selected from manganese, iron, chromium and cobalt, and preferably cobalt. For the avoidance of doubt, reference herein to the term “transition metal” is to the conventional definition of a metal which can accept or donate electrons into its d- or f-orbitals and exhibit a plurality of oxidation states, and includes the lanthanide and actinide series of transition metals. Where the catalyst system comprises copper and one or more additional metals (M), the [M]:[Cu] molar ratio is typically no more than about 500:1, more typically no more than about 100:1, more typically no more than about 20:1, and in one embodiment no more than about 10:1, wherein [M] is the total molar amount of the other metal(s).

The copper and optional additional metal(s) in the copper-containing catalyst is/are typically in the form of one or more metal salt(s). Suitable metal salt(s) include any of those that have been used in the liquid phase oxidation of aromatic carboxylic acid precursors in aliphatic carboxylic acid solvent, e.g. bromides or benzoates (or other aromatic acid salts). It is preferred that the catalyst comprises bromide ions, and preferably that the metal, or at least one and preferably all of the metals present in the catalyst, is/are present as the bromide salt. The catalyst is preferably added to the reaction in pre-prepared form, but it is also possible to form the catalyst within the system by adding reagents which subsequently combine to form the catalyst. For instance, it is possible either to introduce CuBr₂ itself into the system, or to introduce reagents such as copper benzoate and HBr into the system, which combine to form CuBr₂ under the reaction conditions.

In addition to the unexpected activity of copper itself as a catalyst in the oxidation reactions described herein, the inventors have found that the presence of copper in a mixed-metal catalyst results in an unexpected synergistic interaction between the copper and the other metal component(s) of the catalyst. A synergistic interaction is defined herein as the production of a yield which is higher than expected when compared to the sum of the yields for the components making up the catalyst. This unexpected synergistic interaction allows a reduction in the amount of catalyst required, relative to the conventional MnBr₂ catalyst without detriment to the yield and/or selectivity and/or burn of the reaction.

In one embodiment, the catalyst system comprises cobalt and copper, and in this embodiment the Co:Cu molar ratio is preferably no more than about 500:1 preferably no more than about 100:1, preferably no more than about 20:1, and in one embodiment no more than about 10:1. In one embodiment, the Co:Cu molar ratio is at least 1:1, particularly between about 2:1 and 10:1, and particularly between about 2:1 and 9:1, particularly when a low burn is desirable. In one embodiment, the catalyst comprises copper and cobalt, wherein at least one and preferably each metal is present as its bromide. In one embodiment, the metals of the catalyst system consist of copper and cobalt.

In one embodiment of the present invention, hydrogen bromide (HBr) is added to the reaction mixture, particularly when the precursor is p-xylene. Nevertheless, HBr causes corrosion in the system, and so too great an amount is undesirable. The amount of HBr added is preferably such that the molar ratio [HBr]:[M] (where M is the metal ion(s) of the catalyst) is at least 1.0:1, preferably at least 2.0:1, and typically no more than about 50.0:1, more typically no more than about 25.0:1, more typically no more than about 12.0:1, more typically no more than about 6.0:1, and most typically no more than about 4.0:1. In embodiments where HBr is added to the reaction mixture, the addition is effected such that HBr is present in the preferred single homogeneous phase referred to herein, and particularly so that it is present in any location where the metal-containing catalyst is in contact with the oxidant. Thus, contact of at least part, and typically substantially all, of the metal-containing catalyst with the oxidant is effected in the presence of HBr. Thus, HBr is typically introduced into the reaction zone by pre-mixing with the metal-containing catalyst prior to contact with the oxidant or as a separate stream wherein the respective streams comprising metal-containing catalyst, the oxidant/solvent mixture and the HBr are contacted simultaneously. A separate HBr stream may be subjected to pressurisation and, if desired, heating.

The reactor system suitable for performing the process of the present invention may be generally configured as described below.

The oxidation reaction is initiated by heating and pressurising the reactants followed by bringing the heated and pressurised reactants together in a reaction zone. This may be effected in a number of ways with one or both of the reactants being admixed with the aqueous solvent prior to or after attainment of supercritical or near supercritical conditions, such admixture being effected in such a way as to maintain the reactants isolated from one another until brought together in the reaction zone.

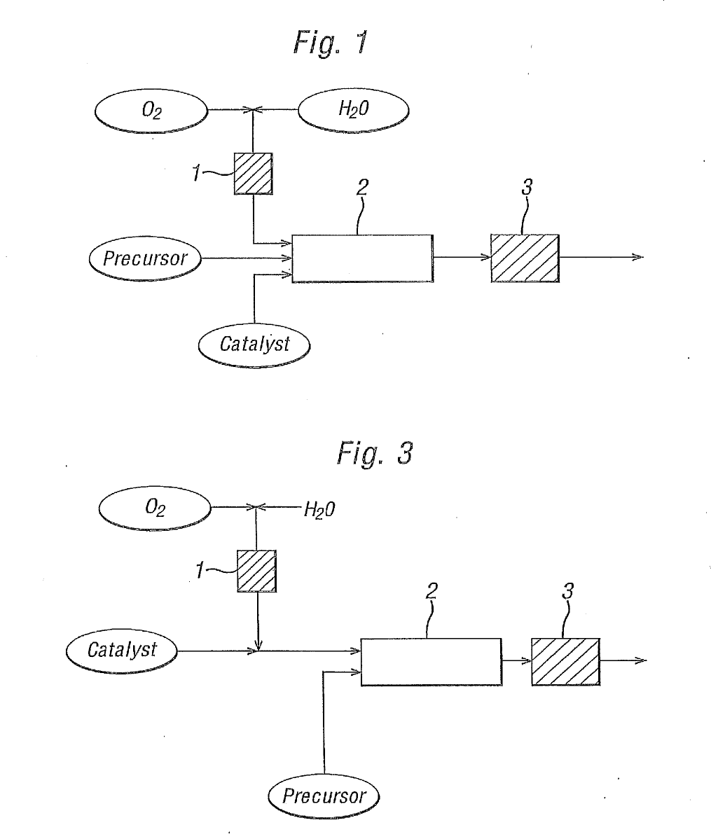

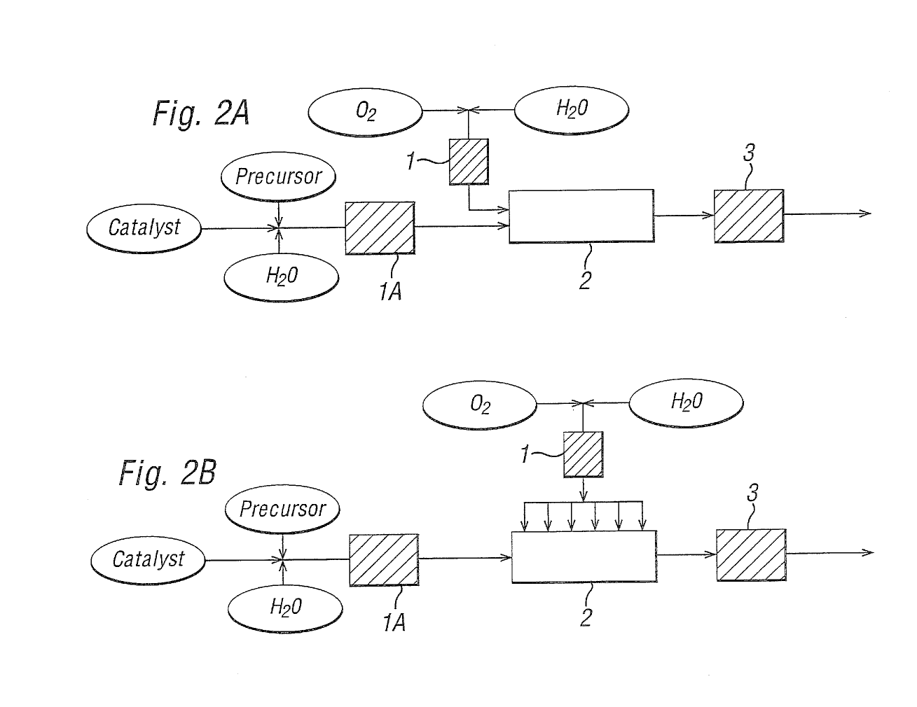

In the continuous process for the production of carboxylic acids described herein, the reactor system is configured such that the contact between the oxidant and at least part, and preferably substantially all, of the precursor is effected in the presence of catalyst. If precursor and oxidant are contacted in the absence of catalyst, the burn of the reaction is unacceptably high. Thus, precursor may be contacted with at least part of the oxidant at the same point in the reactor system as, and contemporaneous with, the contact between the catalyst and at least part of the oxidant, and such a mixing configuration is shown in FIG. 1. Preferably, however, oxidant is contacted with the precursor subsequent to the contact between the catalyst and the precursor, and such arrangements are shown in FIGS. 2A and 2B.

Thus, in Embodiment I, the oxidant is mixed with the aqueous solvent after the latter has been heated and pressurised to secure the supercritical or near supercritical state, with suitable pressurisation and, if desired, heating, of the oxidant prior to mixing with the aqueous solvent. The precursor is subjected to pressurisation and, if desired, heating. The catalyst-comprising component is subjected to pressurisation and, if desired, heating. The separate streams comprising precursor, catalyst and the oxidant/solvent mixture may then be contacted simultaneously. A schematic flow diagram representing Embodiment I is presented in FIG. 1.

In Embodiment II of the invention, the precursor is mixed with the aqueous solvent after the latter has been heated and pressurised to secure the supercritical or near supercritical state, with suitable pressurisation and, if desired, heating, of the precursor prior to mixing with the aqueous solvent. In one arrangement, a homogenous catalyst component after pressurisation and, if desired, heating, is contacted with the aqueous solvent simultaneously with the contacting of the precursor with the aqueous solvent. The oxidant after pressurisation and, if desired, heating, is mixed with aqueous solvent after the latter has been heated and pressurised to secure the supercritical or near supercritical state, and the oxidant/aqueous solvent mixture is then contacted with the mixture comprising the precursor, catalyst and aqueous solvent. Such arrangements are shown in FIGS. 2A and 2B. The mixing configuration of Embodiment II, and particularly the arrangement of FIG. 2B in which the oxidant is introduced at multiple locations across the reaction zone, is particularly preferred in the present invention. It has been found that this configuration results in an unexpectedly low burn for the reaction.

Other configurations of the reactor system are not excluded, provided that the contact between the oxidant and at least part, and preferably substantially all, of the precursor is effected in the presence of catalyst. One such arrangement is shown in FIG. 3, which is a schematic flow diagram for Embodiment III. Thus, in Embodiment III, the oxidant is mixed with aqueous solvent after the latter has been heated and pressurised to secure the supercritical or near supercritical state, with suitable pressurisation and, if desired, heating, of the oxidant prior to mixing with the aqueous solvent. The catalyst is subjected to pressurisation and, if desired, heating. The precursor is subjected to pressurisation and, if desired, heating, and then contacted with the mixture comprising the oxidant and catalyst in the reaction zone.

Contact of the various streams may be effected by way of separate feeds to a device in which the feeds are united to form the preferred single homogeneous fluid phase thus allowing the oxidant and precursor to react. The device in which the feeds are united may for instance have a Y, T, X or other configuration allowing separate feeds to be united in a single flow passage forming the continuous flow reactor, or in some circumstances multiple flow passages forming two or more continuous flow reactors. The flow passage or passages in which the feeds are united may comprise a section of tubular configuration with or without internal dynamic or static mixing elements.

In a preferred embodiment, in-line or static mixers are advantageously used to ensure rapid mixing and homogeneity, for example to promote dissolution of oxidant into the aqueous solvent and the formation of a single phase.

The oxidant feed and the precursor feed may be brought together at a single location or the contact may be effected in two or more stages so that at least part of one feed or of both feeds are introduced in a progressive manner, e.g. via multiple injection points, relative to the direction of flow through the reactor. For instance, one feed may be passed along a continuous flow passage into which the other feed is introduced at multiple points spaced apart lengthwise of the continuous flow passage so that the reaction is carried out progressively. The feed passed along the continuous flow passage may include the aqueous solvent as may the feed introduced at multiple positions.

In one arrangement, the oxidant is introduced to the reaction at two or more locations. Such locations are conveniently so positioned relative to the bulk flow of solvent and reactants through the oxidation zone that oxidant is introduced to the reaction at an initial location and at least one further location downstream of said initial location.

Similarly, the addition of catalyst may be effected in a progressive manner, e.g. via multiple injection points, relative to the direction of flow through the reactor. Where the catalyst system comprises two or more metal-containing species, for instance copper bromide and cobalt bromide, they may be fed together or separately into the reactor, and either at the same location or at different locations in the reactor.

There may be more than one reaction zone in series or in parallel. For instance, where multiple reaction zones in parallel are used, the reactants and solvent may form separate flow streams for passage through the reaction zones and, if desired, the product streams from such multiple reaction zones may be united to form a single product stream. Where more than one reaction zone is used, the conditions, such as temperature, may be the same or different in each reactor. The or each reactor may be operated adiabatically or isothermally. Isothermal or a controlled temperature rise may be maintained by heat exchange to define a predetermined temperature profile as the reaction proceeds through the reactor.

The heat of reaction may be removed from the reaction by heat exchange with a heat-accepting fluid, according to conventional techniques known to those skilled in the art, and described for instance in WO-02/06201-A the disclosure of which techniques is incorporated herein by reference. Conveniently the heat-accepting fluid comprises water.

After traversing the continuous flow reactor and upon completion of the oxidation process, the reaction mixture comprises a solution of aromatic carboxylic acid, which needs to be recovered from the reaction medium. Substantially the entire amount of aromatic carboxylic acid produced in the reaction is in solution at this stage. In the process of the invention, typically at least 80% wt, more usually at least 90% wt, preferably at least 95% wt, more preferably at least 98% wt and most preferably substantially all of the aromatic carboxylic acid produced in the reaction is maintained in solution during the reaction and does not begin to precipitate until the solution leaves the oxidation reaction zone and undergoes cooling. The solution may also contain catalyst, and relatively small quantities of by-products such as intermediates (e.g. p-toluic acid and 4-CBA in the case of terephthalic acid), decarboxylation products such as benzoic acid and degradation products such as trimellitic acid and any excess reactants. The desired product, aromatic carboxylic acid such as terephthalic acid, may be recovered by causing or allowing the aromatic carboxylic acid to crystallise from the solution in one or more stages followed by a solids-liquid separation in one or more stages.

The product stream is subjected to a solids-liquid separation to recover the aromatic carboxylic acid and the mother liquor (which may but need not necessarily contain dissolved catalyst components) is recycled to the oxidation reaction zone. Preferably prior to re-introduction into the oxidation reaction zone, the mother liquor is heated by heat exchange with the product stream thereby cooling the latter.

Any of the reactants may be admixed with the mother liquor recycle stream or separate mother liquor recycle streams prior to re-introduction of the mother liquor into the reaction zone and the mother liquor recycle stream (or at least that fraction or those fractions thereof to be combined with the reactant or reactants) may be heated and pressurised to secure supercritical/near supercritical conditions before being admixed with the reactant or respective reactant.

The invention will now be described further by way of example only with reference to the accompanying drawings.

Referring to FIG. 1, dioxygen, after pressurisation, is mixed with water after the water has been heated and the mixture pressurised and optionally further heated in preheater 1 to achieve the supercritical state. The precursor and catalyst are added, after pressurisation, to the O₂/water stream at the beginning of or immediately before the reactor 2 and the mixture passed through the reactor. Upon exiting the reactor, the stream is cooled and depressurised at the back-pressure regulator 3. The products are carried out in a stream of cooled water.

Referring to FIGS. 2A and 2B, the precursor and catalyst after pressurisation are added to water after the water has been pressurised and optionally heated. The mixture is optionally further heated in preheater 1A to achieve the supercritical state. The dioxygen gas, after pressurisation is mixed with water at a supercritical state and optionally further heated in preheater 1. In FIG. 2A, the streams are mixed at the beginning of or immediately before the reactor 2 and the mixture passed through the reactor. In FIG. 2B, the O₂/water stream is added to the reactor in a progressive manner at multiple injection points. Upon exiting the reactor, the stream is cooled and depressurised at the back pressure regulator 3. The products are carried out in a stream of cooled water.

FIG. 3 corresponds to FIG. 1 wherein the catalyst and oxidant are mixed prior to contact of either stream with the precursor. The dioxygen gas after pressurisation is mixed with water at a supercritical state and optionally further heated in preheater 1.

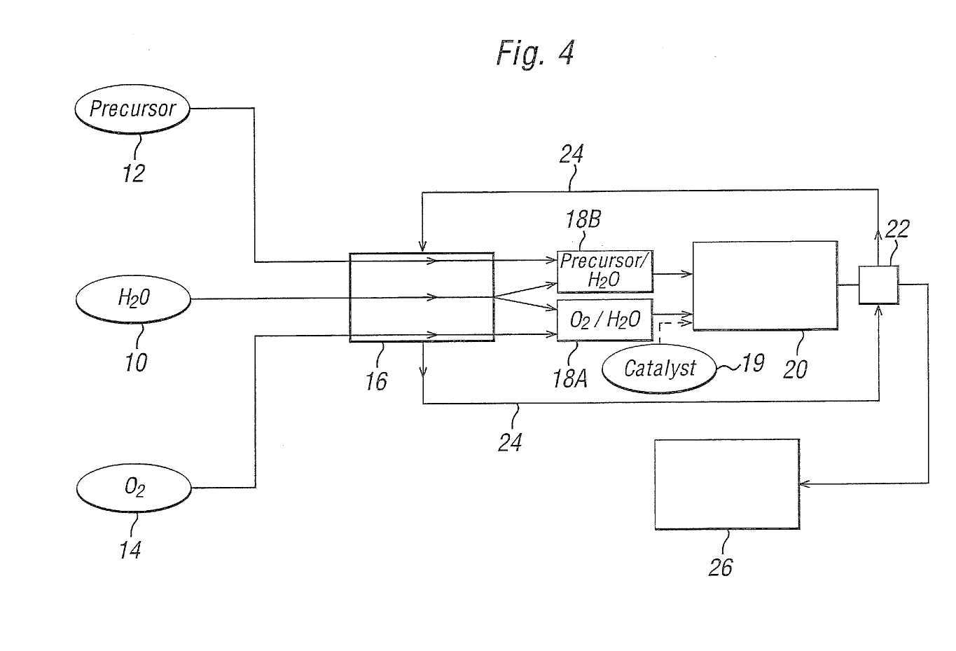

Referring to FIG. 4, feedstock components comprising water, precursor (e.g. paraxylene in the process for the production of terephthalic acid) and dioxygen gas are pressurised to operating pressure and continuously supplied from respective sources 10, 12 and 14 through a preheater 16 where the components are heated to a temperature of 300 to 480° C., more preferably 330 to 450° C., typically from about a lower limit of about 350 to 370° C. to an upper limit of about 370 to about 420° C., the pressure and temperature being selected in order to secure supercritical or near supercritical conditions. Part of the heat used to preheat the feedstock components may be derived from the exotherm produced in the course of the subsequent reaction between the precursor and the oxidant. Heat from other sources may be, for example, in the form of high pressure steam and/or heating may be effected by direct fired heating of the water stream. The heat of reaction may be recovered in any suitable manner, e.g. by means of heat exchange between the fluid following reaction and a suitable heat-accepting fluid such as water. For instance, the heat-accepting fluid may be arranged to flow in heat exchange relation, counter-currently and/or co-currently, with the reactants and solvent passing through the reaction zone. The passage or passages along which the heat-accepting fluid flows in traversing the reaction zone may be external to the reaction zone and/or may extend internally through the reaction zone. Such internally extending flow passage(s) may for instance extend generally parallel with and/or transversely of the general direction of flow of the reactant/solvent through the reaction zone. For example, the heat-accepting fluid may traverse the reaction zone by passage through one or more coiled tubes located within the interior of the reactor. The enthalpy of reaction can be used to recover power via a suitable power recovery system such as a turbine; for instance the heat-accepting fluid, e.g. water, can be used to raise high pressure saturated steam at for example temperature and pressure of the order of 300° C./100 bara which, in turn, can be superheated by external heat and fed to a high efficiency condensing steam turbine to recover power. In this way, the reactor can be maintained at an optimum temperature and effective energy efficiency can be achieved. In an alternative approach, the reactor may be operated under adiabatic conditions and a suitably high rate of water flow through the reaction zone may be employed in order to constrain the temperature rise across the reactor in operation. If desired, a combination of both approaches may be used, i.e. recovery of the enthalpy of reaction via a heat-accepting fluid coupled with a suitable water flow rate through the reaction zone.

Following heating of the feedstock components, oxygen is mixed with water which, as a result of preheating and pressurisation, will be under supercritical or near supercritical conditions and hence capable of solubilising the feedstocks. In the embodiment illustrated in FIG. 4, oxygen and water are mixed in premixer 18A. The precursor is also mixed with water in premixer 18B. Of course, the precursor could also be separately premixed with water prior to entry into the preheater 16.

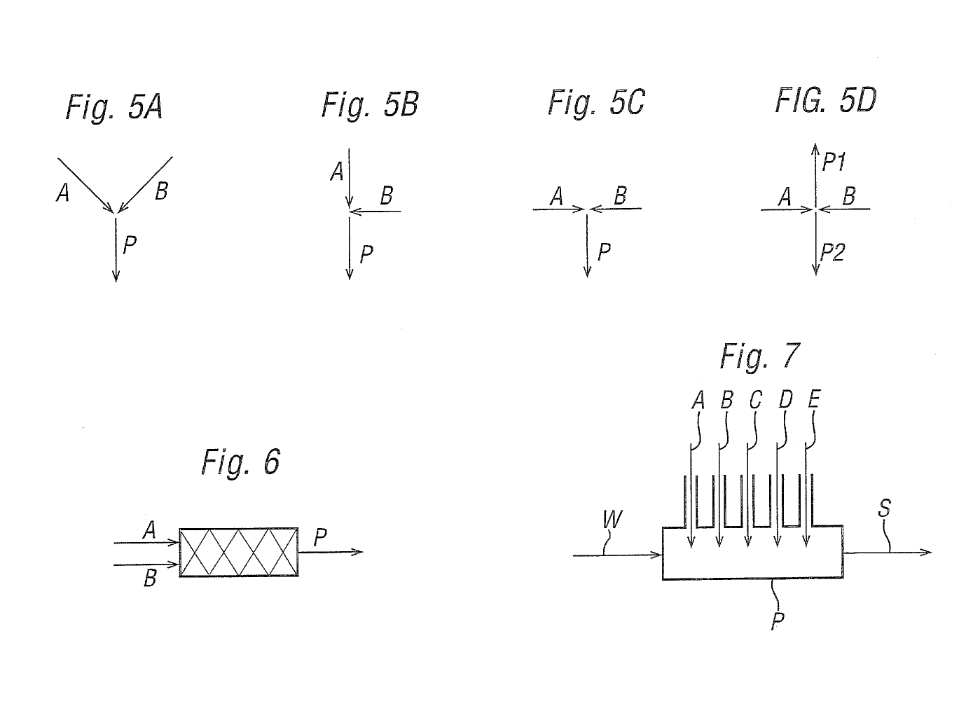

The premixer (or premixers where premixing of each reactant and water is undertaken) may take various forms such as Y, L or T piece, double T configurations or a static mixer, as illustrated in FIGS. 5A, 5B, 5C, 5D and 6 respectively. In FIGS. 5A to 5D and 6, reference A depicts the preheated water supply to the premixer, B depicts the reactant (precursor or oxygen) and P depicts the resulting mixed stream. In the double T configuration of FIG. 5D, two mixed streams are produced P1 and P2. These may either be passed through separate continuous flow reactors or be combined into a single stream and then passed through a single continuous flow reactor. An X piece configuration may also be used, as known to those skilled in the art. It will also be appreciated that any suitable mixing apparatus may be used in the present invention. It will further be appreciated that the mixing apparatus referred to above are for use in a continuous process apparatus. In a batch system, there is of course no continuous flow and therefore no specific flow-related mixing requirements. In a continuous vessel reactor, reactants can also be fed into the vessel independently.

It will be appreciated that instead of premixing one or each reactant with water prior to introduction into the reaction zone, the reactants and water may be introduced separately into the reaction zone and mixed within the reaction zone with the aid of some form of mixing arrangement (e.g. a static mixer) whereby substantially all mixing of the components occurs within the reaction zone.

The homogeneous catalyst is added as a solution from source 19 to the premixed oxygen/water stream at the same time as the precursor is added to the premixed oxygen/water stream either immediately prior to entering the reactor or at the beginning of the reactor (i.e. as shown in FIG. 1).

Following preheating and premixing, the feedstock components are combined in a reaction zone 20 to form a single homogeneous fluid phase in which the reactants are brought together. The reaction zone 20 may consist of a simple mixer arrangement in the form of a tubular flow reactor, e.g. a pipe of a length which, in conjunction with the flow rate of the combined reactants, provides a suitable reaction time so as to secure conversion of, for example, paraxylene to terephthalic acid with high conversion efficiency and low 4-CBA content.

The reactants may be combined in a progressive manner by injecting one reactant into a stream containing the other reactant at multiple points along the length of the reactor. One way of implementing a multiple injection arrangement is shown in the continuous flow reactor of FIG. 7 in which the reactor is constituted by a pipe or vessel P. In an embodiment wherein a premixed oxygen/water stream is added to a premixed precursor/water stream, a premixed precursor/supercritical or near supercritical water stream W is supplied to the upstream end of pipe or vessel P. Water stream W would also contain the homogeneous catalyst. The stream passes through the reactor pipe or vessel P and at a series of locations spaced at intervals along the length of the pipe or vessel P, preheated and compressed oxygen dissolved in supercritical or near supercritical water is supplied via multiple injection passages A to E to produce a product stream S comprising carboxylic acid product (e.g. terephthalic acid) in supercritical or near supercritical aqueous solution. In this manner, the oxygen necessary to effect complete oxidation of, for example, paraxylene to terephthalic acid is injected progressively with the aim of controlling oxidation and minimising side reactions and possible burning of paraxylene, terephthalic acid or terephthalic acid intermediates.

Referring now back to FIG. 4, following the reaction to the desired degree, the supercritical or near supercritical fluid is passed through a heat exchanger 22 through which heat exchange fluid is circulated via closed loop 24 so that heat can be recovered for use in the preheater 16. One scheme (not shown) for post-reaction cooling of the carboxylic acid product solution involves the use of heat exchanger networks to cool the stream to subcritical temperatures, e.g. of the order of 300° C. to retain the carboxylic acid product in solution and thereby reducing fouling of heat exchange surfaces, followed by use of a train of flashing crystallisers (similar to those employed in conventional terephthalic acid purification by hydrogenation) to cool and precipitate the carboxylic acid product.

The cooled solution is then supplied to a product recovery section 26 in which the carboxylic acid is precipitated from the solution. Any suitable method of product recovery known to those skilled in the art may be used, for instance those disclosed in WO-02/06201-A or the Applicant's co-pending applications derived from United Kingdom patent applications 0621970.3 and 0621968.7, the disclosures of which are incorporated herein by reference. Although in general, it will be desirable to produce carboxylic acid product, such as terephthalic acid, which is sufficiently pure to render further purification unnecessary (e.g. by oxidation and/or hydrogenation of an aqueous solution of terephthalic acid to convert 4-CBA to terephthalic acid or to para-toluic acid, as the case may be), we do not exclude the possibility of carrying out such purification subsequent to the supercritical or near supercritical water oxidation reaction.

Following recovery of the aromatic carboxylic acid product, at least part of the aqueous mother liquor may be recycled for re-use in the oxidation reaction, e.g. by admixture with fresh water and/or the reactants. However, if the recycled mother liquor contains catalyst components, it is preferably not added to the O₂/water stream prior to addition of precursor. The amount recycled will usually be a major fraction of the recovered mother liquor, with a purge being taken in order to reduce standing concentrations of by-products in the process. The purge stream may be treated to recover its catalyst content where applicable and its organic content.

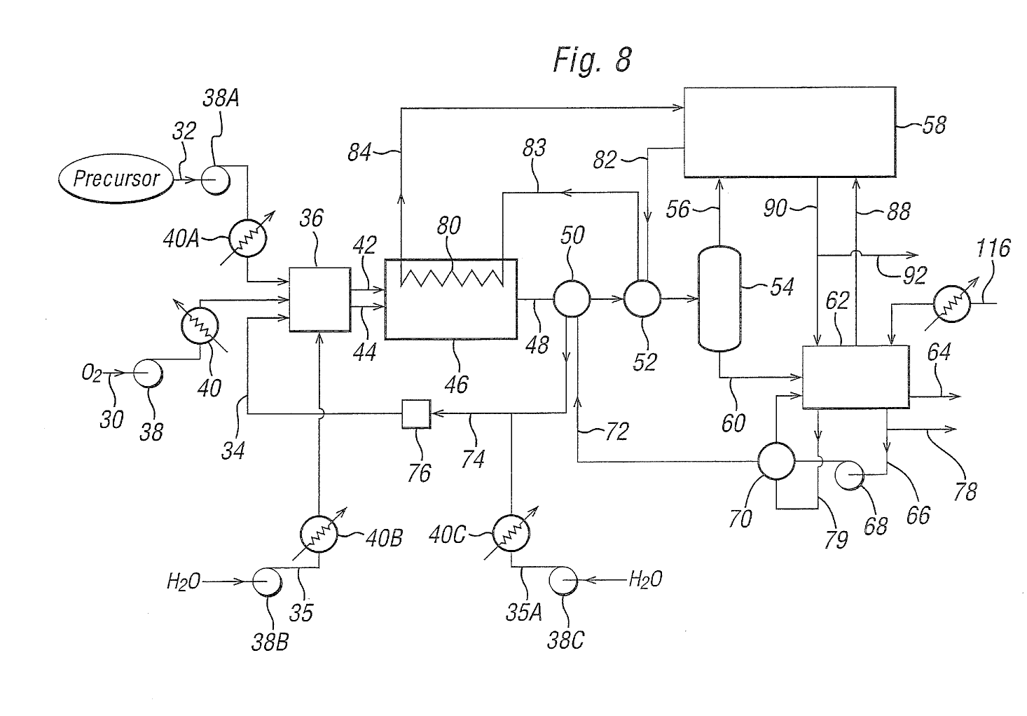

Referring now to FIG. 8, in this embodiment oxygen (line 30), liquid precursor (e.g. paraxylene in the case of the process for the production of terephthalic acid) (line 32) and water (line 34) are supplied to a mixing unit 36. The oxygen and precursor supplies are pressurised by pumps 38, 38A and heated to elevated temperature, for example by high pressure steam, in heat exchangers 40, 40A. The mixing unit 36 is configured, as shown in FIG. 4, to mix the reactants with the water supply to produce two streams 42, 44, one stream comprising a water/precursor mixture and the other stream comprising oxygen dissolved in water, which are fed to a continuous flow reactor 46 in the form of a pipe in which the streams are mixed, e.g. by an unshown static mixing arrangement within the pipe, to initiate the reaction. The homogeneous catalyst as a solution in water may be added either into the precursor/water stream 42 immediately prior to entering the reactor, or on combination of streams 42 and 44 at the beginning of or immediately before the reactor, using rapid mixing, for example by the use of a static mixer or similar device.

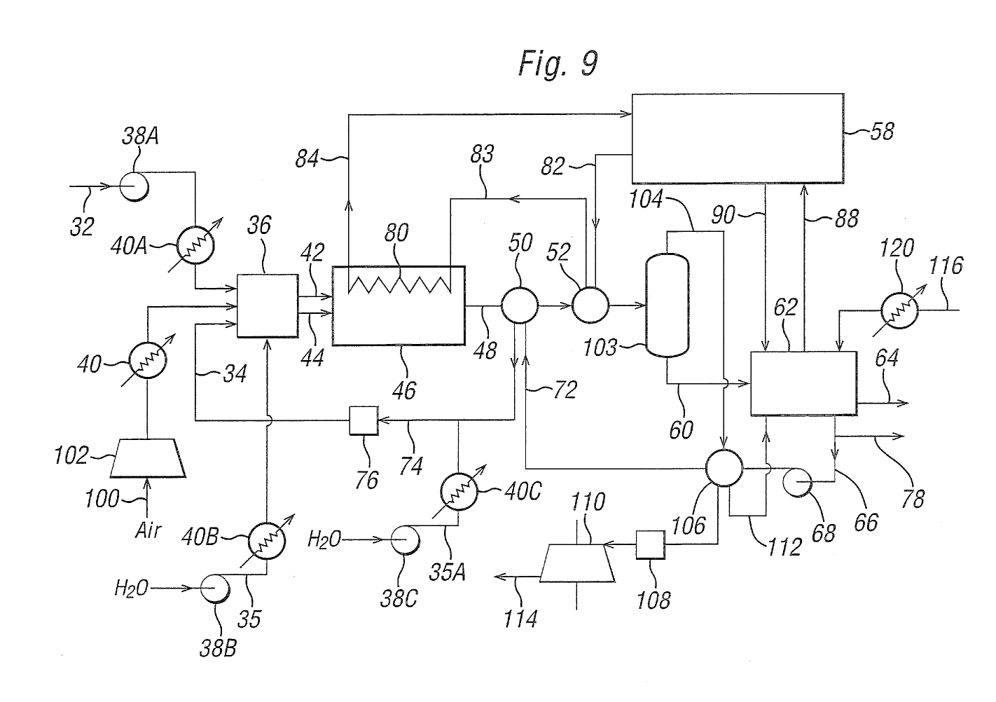

The supply of fresh make-up water to the system may be effected at various points. One of the most convenient points is upstream of the main pressurisation pump 68, for instance via line 116 which is described in more detail below in relation to FIG. 9. Water may also be fed after pressurisation in pump 38C and heating in heat exchanger 40C via line 35A into line 74, or prior to the exchangers (50,70). Alternatively, water may be fed, after pressurisation in pump 38B and heating in heat exchanger 40B independently into the preheater 36 via line 35.

Following reaction under supercritical or near supercritical conditions, the product stream 48 in the form of a solution of reaction product(s) (plus small amounts of unreacted reactants, intermediates etc) is cooled by passage through heat exchangers 50 and 52 and may be optionally flashed down to a lower pressure and temperature in flash vessel 54. The means of effecting such a step at this point or in the product recovery section 62 may involve known devices, singly or in multiples, but should be configured to avoid deposition of solids, by means such as localised heating, as known to those skilled in the art. Thus, as the stream from reactor 46 is passed through heat exchangers 50 and 52, the temperature of the stream is monitored and controlled so that the product does not precipitate; precipitation should not occur until flash vessel 54. A substantial amount of steam and some gaseous components such as nitrogen, oxygen, carbon oxides are supplied via line 56 to an energy recovery system 58 while the terephthalic acid solution is supplied via line 60 to a product recovery section 62.

In FIG. 8, the carboxylic acid crystals recovered are supplied via line 64 to a drier (not shown) or to the direct production of polyester. Where the solids-liquid separation is carried out under elevated pressure conditions, the crystals are conveniently let down to atmospheric pressure using a suitable device (e.g. as disclosed in International Patent Application No. WO-A-95/19355 or U.S. Pat. No. 5,470,473) before being transferred to drying equipment. The mother liquor from the solids-liquid separation is recovered via line 66, repressurised by pump 68 and recycled to the mixer unit 36 via heat exchanger 70, line 72, heat exchanger 50, line 74, start-up/trim heater 76 and line 34. Thus, under steady state operating conditions, the recycled mother liquor may contribute to the source of water for supply to the reactor 46 as well as a vehicle for the recycle of catalyst to the process. The mixture unit 36 is configured such that, where the recycled mother liquor may contain catalyst, i.e. homogeneous catalyst, the recycled mother liquor is preferably mixed with the precursor stream rather than the oxidant stream since the addition of catalyst to oxidant is preferably contemporaneous with the addition of precursor to oxidant. Thus, where the recycled mother liquor contains catalyst, the mixture unit is configured such that the oxidant stream 30 may be mixed with fresh water from line 35. Similarly, additional catalyst, as required, may be added to the mother liquor in line 34, or directly to the reaction zone 46.

Because water is generated in the course of the reaction, a water purge is taken from the system. This may be effected in several ways; for instance, the purge may be taken via line 78 or from a suitable flash condensate (for example as will be described below in connection with the energy recovery system). The latter may be more advantageous as it will be somewhat less contaminated with organics than a purge from the mother liquor recovered via line 66. The purge however recovered may be passed to effluent treatment, e.g. aerobic and/or anaerobic processing.

In the heat exchanger 70, the temperature of the mother liquor is increased by about 30 to 100° C., through heat transfer from steam flashed from one or more of the crystallisation stages, e.g. the first stage highest pressure and temperature crystalliser vessel. The flash (line 79) used for this purpose may, following passage through the heat exchanger 70, be returned as condensate to the product recovery section for use as wash water in washing the carboxylic acid product filter cake produced by solids-liquid separation. In the heat exchanger 50, the temperature of the mother liquor is increased still further, for instance by about 100 to 200° C., as a result of heat transfer from the high temperature product stream 48 from the reactor 46. In this manner, the product stream is subjected to cooling while significantly increasing the temperature of the mother liquor recycle stream. The trim/start-up heater 76 serves to boost the temperature of the mother liquor recycle stream, if necessary, to secure supercritical or near supercritical conditions. Under steady state operation of the process such boost may be optional since the mother liquor may be rendered supercritical or near supercritical following passage through the heat exchanger 50. The heater 76 may not therefore be necessary under steady state conditions and may be deployed purely for start-up operation, initially using pressurised water from a source other than mother liquor. In this embodiment, the water solvent is rendered supercritical or near supercritical prior to mixing with one or both reactants. However, it will be understood that raising of the temperature to secure the desired supercritical or near supercritical conditions may be effected prior to, during and/or following the mixing stage.

In the embodiment of FIG. 8, the heat of reaction generated in the course of reacting the precursor with oxygen is removed at least in part by heat exchange with a heat-accepting fluid, preferably water, which is passed through the interior of the reactor 46 by means of a coiled tube 80 or a series of generally parallel tubes (as in a tube in shell heat exchanger design) or the like. The water employed is pressurised and heated to a temperature sufficiently high that, at the external surface of the conduit or conduits 80 conducting the water through the reactor, localised cooling which could otherwise cause precipitation of components, such as terephthalic acid, in the reaction medium is avoided. The water for this purpose is derived from the energy recovery system 58. Thus, in FIG. 8, water at elevated pressure and temperature is supplied via line 82 to heat exchanger 52 where it is used to cool the product stream further following passage through the heat exchanger 50. The water then passes via line 83 through the conduit(s) 80 with consequent raising of high pressure, high temperature steam which is fed to the energy recovery system 58 via line 84.

The energy recovery system 58 is also supplied with steam flashed from one or more stages of the crystallisation train. This is depicted by line 88. This steam may for example be used to preheat the water supplied via line 82 to the heat transfer conduit(s) 80. Condensate resulting from processing of the steam feeds supplied to the energy recovery system 58 may be passed via line 90 to the product recovery section for use for example in washing the terephthalic acid filter cake produced in the solids-liquid separation. A water purge 92 may be taken from line 90 if desired, with the advantage that a purge taken at this point will be less contaminated than a purge taken from the mother liquor via line 78.

In FIG. 8, the reactant(s) are shown as being introduced into the recycled mother liquor after the mother liquor has been heated by heat exchange with the product stream in heat exchanger 50. In a modification, a reactant may be admixed with the mother liquor recycle stream upstream of the heat exchange with the product stream. Where both reactants are so admixed with the mother liquor recycle stream, the latter is split into separate streams with which the reactants are respectively admixed so that the reactants are maintained isolated from each other until brought together for reaction. It will also be understood that the embodiment of FIG. 8 may be modified in the manner indicated in FIG. 7 by introducing one or even both of the reactants via multiple injection points along the flow path of the reaction medium so that the one or both reactants are introduced to the reaction progressively.

In the energy recovery system 58, various heat recovery processes may be carried out in order to render the process energy efficient. For instance, the high pressure steam raised following passage of water through the conduit(s) 80 may be superheated in a furnace supplied with combustible fuel and the superheated steam may then be passed through one or more steam condensing turbine stages to recover power. Part of the high pressure steam may be diverted for use in preheating the reactants (heat exchangers 40, 40A and 40B) or for preheating stream 82 where this is necessary to effect a system of high thermal efficiency. The condensed water recovered from the turbine stages and from the heat exchangers 40, 40A and 40B may then be passed through a train of heating stages in order to preheat the water for recirculation to the reactor 46 via heat exchanger 52 thus forming a closed loop with make-up water being added as needed. The heating stages typically comprise a cascade of heat exchangers by means of which the recirculating water flow returning to the reactor 46 is progressively raised in temperature. In some heating stages, the heat-donating fluid may be constituted by the flash steam derived at different pressures and temperatures from different stages of the crystallisation train. In other heating stages, the heat-donating fluid may be combustion gases rising in the furnace stack associated with the furnace used to superheat the high pressure steam supplied via line 84.

The embodiment of FIG. 8 employs substantially pure oxygen as the oxidant. FIG. 9 illustrates a similar embodiment but using a supply of compressed air (which may be oxygen enriched) as the oxidant. The embodiment of FIG. 9 is generally similar to that of FIG. 8 and those parts which function in generally the same way are depicted by the same reference numerals in both Figures and will not be described further below unless the context requires otherwise. As shown, the air supply 100 is supplied via an air compressor 102. As a result of using air, a substantial amount of nitrogen is introduced into the process and must therefore be appropriately handled. In this case, the product stream following passage through the heat exchangers 50 and 52 is flashed down in flash vessel 103 to a lower temperature to condense water to a greater extent than in the embodiment of FIG. 8 thereby reducing the water content of the overheads. As described in relation to FIG. 8, temperature of the product stream through the heat exchangers 50 and 52 is controlled such that precipitation of product occurs only in flash vessel 103. The overheads stream is supplied via line 104, heat exchanger 106 and fuel-fired heater 108 to a gas turbine 110. The overheads stream is passed through heat exchanger 106 in order to transfer heat to the mother liquor recycle stream while knocking out further water which can be passed to the product recovery section 62 via line 112 for use, for example, as wash water. For reasons of energy efficiency, it is desirable to heat the gaseous overheads stream to a high temperature before introduction into the turbine 110, hence the reason for heating the overheads stream by means of heater 108. There may be more than one gas turbine stage, in which case the overheads stream will be heated to an elevated temperature upstream of each such turbine stage. Line 114 depicts the overheads stream exiting the turbine 110 at low pressure and temperature. Where the oxidation process leads to the generation of species such as carbon monoxide etc. which are undesirable, for example for corrosion and/or environmental reasons, provision may be made for treating the overheads stream to reduce/eliminate such components before or after passage through the turbine 110 and/or discharge. Such treatment may comprise subjecting the overheads stream to catalytic combustion and/or scrubbing with a suitable reagent, e.g. an alkaline scrubbing liquor. The turbine 110 may be mechanically coupled with the air compressor so that the latter is driven by the turbine.

In the embodiment of FIG. 9, water exits the system via the overheads stream. At least part of this water may be recovered if desired and recirculated for use for example as wash water in the product recovery section 62. Alternatively or additionally, make-up water may be supplied via line 116 to the product recovery section to compensate for the water lost in handling the large volumes of nitrogen as a result of compressed air usage. Such make-up water may be preheated and used as wash water, preheating being effected for example by diverting part of the flash streams (collectively depicted by reference numeral 88) via line 116 to heat exchanger 120 and returning the water condensed from the flash stream to the product recovery section 62 as wash water.

Although the invention has been described mainly with reference to para-xylene as a precursor for terephthalic acid, it will be appreciated that other precursors may be employed instead or in addition to para-xylene for the production of the corresponding carboxylic acid, and such precursors include ortho-xylene, meta-xylene, 4-tolualdehyde, 4-toluic acid and 3-methylpyridine. As noted above, the invention is also applicable to the production of other aromatic carboxylic acids such as isophthalic acid, phthalic acid, trimellitic acid and naphthalene dicarboxylic acid from the corresponding alkyl aromatic compounds (preferably the methyl compounds) or other precursors. The invention is further illustrated below by the following non-limiting Examples.

### EXAMPLES

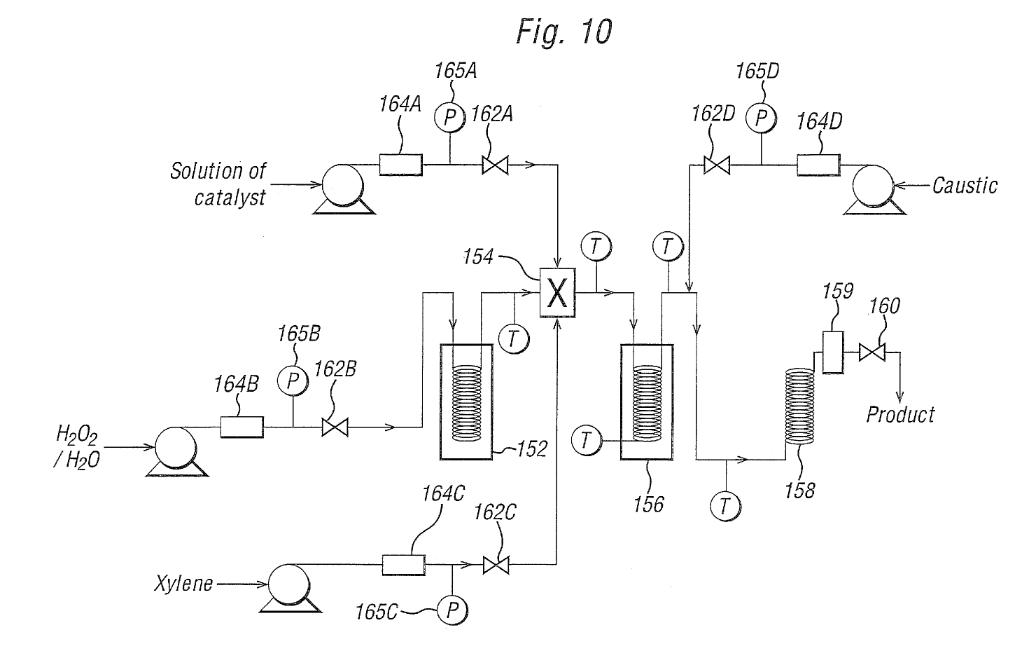

Experimental work was carried out on a laboratory scale by the continuous oxidation of alkylaromatics by O₂ in near critical or supercritical water at about 330-380° C. and 230 to 250 bara with a catalyst solution (as detailed below). The exotherm was minimised by using relatively dilute solutions (0.4%-2.0% organic w/w). The basic configuration of the system is as set out in FIG. 1. A more detailed illustration of the system used in these laboratory scale experiments is shown in FIG. 10.

O₂ originates from heating an H₂O₂/H₂O mixture in excess of 400° C. in the preheater 152. The H₂O₂ decomposes to liberate O₂. The O₂/H₂O fluid then passes through the cross-piece 154, where it is contacted with the alkylaromatic and catalyst solution, fed in from their own pumps. The reaction mixture is passed through the reactor 156. At the end of the reactor, the reaction is quenched by caustic solution added with a pump. Sufficient caustic is used to attain a pH of >12 in the discharge stream. At this pH the product acid (e.g. terephthalic acid) and other intermediates (e.g. p-toluic acid, 4-carboxybenzaldehyde (4-CBA)) are in solution as their sodium salts and CO₂ is captured in solution as sodium carbonate.

Other components labelled in FIG. 10 are as follows: cooling coil 158; 0.5 μm filter 159; back-pressure regulator 160; valves 162 A to D; non-return valves 164 A to D; pressure transducers 165 A to D; thermocouple T (the aluminium heater blocks of preheater 152 and reactor 156 also contain thermocouples, not shown). The pumps were Gilson 305, 306 and 303; the back pressure regulator was obtained from Tescom.

Maximum corrosion occurs in the region of the crosspiece 154 where O₂, feedstock and the catalyst solution meet, particularly at the incoming unheated catalyst feed pipe. Hastelloy was used for the catalyst feed-pipe and for the reactor, and 316 stainless steel for the other components.

Before each run, the apparatus is hydrostatically pressure-tested when cold, and is then heated with a flow of pure water (5-10 mL/min). Once the operating temperature has been reached, H₂O₂/H₂O is fed and the pumps for alkylaromatic and catalyst are started, typically in that order. The residence time for each run remains constant and is typically up to about 1 minute, but in most cases about 0.1-20 seconds.

The products, intermediates and (non gaseous) by products were quantified by HPLC using a Hewlett Packard 1050. For example, when using p-xylene (p-X) feed, typical components were terephthalic acid (TA), p-toluic acid (p-Tol), 4-carboxybenzaldehyde (4CBA) and benzoic acid (BA). The carbon dioxide (CO₂) from burning of the aromatic was quantified by pH titration of the cooler discharge stream with dilute hydrochloric acid to determine its sodium carbonate content.

Results are expressed in the tables in % yield of product from alkylaromatic fed and % of alkylaromatic fed converted to CO₂. Intermediates and byproducts are expressed either as % yields or as % selectivities defined as:

$${S}_{X} = \frac{100 {Y}_{X}}{\sum {Y}_{Ar}}$$

### Where:

S_{X} is the % selectivity of component X
Y_{X} is the % yield of component X
ΣY_{Ar} is the sum of the yields of aromatic components

### Examples 1-22

Experiments were conducted using the following experimental conditions:

Temperature=approx. 380° C.; Pressure=approx. 230 bara

Flow rate of catalyst=4.0 mL/min.

Flow rate of p-xylene=0.061 mL/min

Flow rate oxidant (H₂O₂ in H₂O)=8.1 mL/min. (providing an amount of [O₂] as aqueous H₂O₂ of 0.276 mol.L⁻¹ (1.5 molar equivalents of the stoichiometry required for complete oxidation of the organic precursor to the aromatic acid, the molar ratio for which in the case of p-xylene is 3O₂/organic)).

### Analysis of the Data

The data are presented in Tables 1-4. The data in table 1 demonstrate the surprising superiority of copper-based catalysts in super-critical water oxidation reactions when compared with the conventional manganese or cobalt-based catalysts, in terms of both yield and selectivity.

The data in table 2 demonstrate the improved yields and selectivities when copper and cobalt are combined as catalysts, which at certain metal ratios exhibit lower burn. The data indicate a preferred range for the Co/Cu ratio in a combined cobalt bromide-copper bromide catalyst system for maximising yield, which is between about 5:1 and 100:1 (Co:Cu). Moreover, Co:Cu ratios between about 1:1 and 9:1 exhibit surprisingly reduced burn, with low 4-carboxyaldehyde and p-toluic acid. In one embodiment, therefore, the Co:Cu ratio is preferably in the range of about 1:1 to 10:1.

The data in Table 3 demonstrate the effect of additional metals in the copper catalyst system, and the particularly advantageous nature of the cobalt-copper-bromide catalyst.

The data in Table 4 demonstrate the effect of hydrogen bromide acid in the system. The acid enables high yield with superior selectivity and burn compared to the case without acid. Examples 19-22 illustrate that to achieve the full improvement requires the presence of the both the acid and the additional bromide.

### Examples 23-28

Experimental conditions were the same as in Examples 1-22 except that:

Flow rate of p-xylene=0.28 mL/min

Pressure=approx. 250 bara

Flow rate oxidant (H₂O₂ in H₂O)=8.1 mL/min. (providing an amount of [O₂] as aqueous H₂O₂ of 1.26 mol.L⁻¹ (1.5 molar equivalents of the stoichiometry required for complete oxidation of the organic precursor to the aromatic acid, the molar ratio for which in the case of p-xylene is 3O₂/organic)).

The data in Table 5 demonstrate that increase in catalyst concentration increases yield of terephthalic acid and reduces burn to carbon dioxide. It also further demonstrates the increased activity and reduced burn achieved with a copper-cobalt catalyst.

### Examples 29-30

Experimental conditions were the same as in Examples 1-22 except that:

The alternative feedstocks 4-methylanisole and o-xylene were used at concentration 0.4% w/w.

The temperature for the 4-methylanisole oxidation was subcritical as noted in Table 6a. The hydrogen peroxide concentration was adjusted as necessary to maintain 1.5 molar equivalents of the stoichiometry required for complete oxidation of the organic precursor to the aromatic acid. These stoichiometric ratios are 1.5 and 3.0 moles O₂/mole organic for 4-methyl anisole and o-xylene respectively.

The data in Table 6a and Table 6b exemplify the use of the copper-cobalt based catalyst system for the water based oxidation of 4-methylanisole and o-xylene respectively.

TABLE 1
p-xylene oxidation with metal bromides as catalyst

| | | Catalyst | | | | | Selectivity (%) | | | | Yield (%) | |
|---|---|---|---|---|---|---|---|---|---|---|---|---|
| Run | Feed | type | Catalyst | [Br], M | Br/M | [metals], M | TA | p-Tol | 4-CBA | BA | TA | CO2 |
| Control 1 | p-X | none | none | 0 | — | 0 | 8.4 | 73.3 | 4.7 | 1.9 | 0.6 | 14.3 |
| Comp. Ex. 1 | p-X | Co/Br | CoBr₂ | 0.0051 | 2.00 | 0.0026 | 21.8 | 56.4 | 13.9 | 2.6 | 8.8 | 16.6 |
| Comp. Ex. 2 | p-X | Mn/Br | MnBr₂ | 0.0051 | 2.00 | 0.0026 | 51.3 | 34.9 | 6.3 | 6.0 | 36.1 | 18.4 |
| Example 1 | p-X | Cu/Br | CuBr₂ | 0.0051 | 2.00 | 0.0026 | 85.7 | 5.9 | 1.3 | 7.3 | 54.0 | 26.1 |

TABLE 2
Co/Cu/Br catalysts; variation of Co/Cu ratio.

| | | Catalyst | | | | | Selectivity (%) | | | | Yield (%) | |
|---|---|---|---|---|---|---|---|---|---|---|---|---|
| Run | Feed | type | Catalyst | [Br], M | [metals], M | Co/Cu | TA | p-Tol | 4-CBA | BA | TA | CO2 |
| Comp. Ex. 1 | p-X | Co/Br | CoBr2 | 0.0052 | 0.0026 | | 21.8 | 56.2 | 13.9 | 2.6 | 8.8 | 16.6 |
| Example 2 | p-X | Co/Cu/Br | 0.0022/0.9978 | 0.0052 | 0.0026 | 454 | 26.6 | 55.3 | 9.7 | 3.3 | 12.8 | 23.8 |
| | | | CuBr2/CoBr2 | | | | | | | | | |
| Example 3 | p-X | Co/Cu/Br | 0.01/0.99 CuBr2/CoBr2 | 0.0052 | 0.0026 | 99.0 | 87.6 | 3.0 | 1.0 | 8.4 | 55.1 | 24.6 |
| Example 4 | p-X | Co/Cu/Br | 0.03/0.97 CuBr2/CoBr2 | 0.0052 | 0.0026 | 32.3 | 88.3 | 2.5 | 1.1 | 8.1 | 56.8 | 24.6 |
| Example 5 | p-X | Co/Cu/Br | 0.05/0.95 CuBr2/CoBr2 | 0.0052 | 0.0026 | 19.0 | 91.5 | 0.3 | 0.8 | 7.4 | 64.2 | 22.5 |
| Example 6 | p-X | Co/Cu/Br | 0.1/0.9 CuBr2/CoBr2 | 0.0052 | 0.0026 | 9.0 | 92.0 | 0.0 | 0.5 | 7.6 | 66.4 | 21.9 |
| Example 7 | p-X | Co/Cu/Br | 0.15/0.85 CuBr2/CoBr2 | 0.0052 | 0.0026 | 5.7 | 92.1 | 0.0 | 0.0 | 7.9 | 61.7 | 25.9 |
| Example 8 | p-X | Co/Cu/Br | 0.33/0.66 CuBr2/CoBr2 | 0.0052 | 0.0026 | 2.0 | 89.7 | 1.9 | 0.0 | 8.4 | 51.6 | 26.3 |
| Example 9 | p-X | Co/Cu/Br | 0.5/0.5 CuBr2/CoBr2 | 0.0052 | 0.0026 | 1.0 | 88.4 | 1.7 | 0.4 | 9.3 | 50.4 | 27.0 |
| Example 10 | p-X | Co/Cu/Br | 0.66/0.33 CuBr2/CoBr2 | 0.0052 | 0.0026 | 0.5 | 85.3 | 5.8 | 0.3 | 8.4 | 50.7 | 29.2 |
| Example 1 | p-X | Cu/Br | CuBr2 | 0.0052 | 0.0026 | 0.0 | 85.7 | 5.9 | 1.3 | 7.3 | 54.0 | 26.1 |

TABLE 3
Combinations of Cu/Br or Co/Cu/Br with other metals. Entries are ranked with increasing TA
yield. Examples already described in earlier Tables are included for comparison purposes.

| | | Catalyst | | | | Selectivity (%) | | | | Yield (%) | |
|---|---|---|---|---|---|---|---|---|---|---|---|
| Run | Feed | type | Catalyst | [Br], M | [metals] M | TA | p-Tol | 4-CBA | BA | TA | CO2 |
| Example 11 | p-X | Cu/Mn/Br | CuBr₂/MnBr₂ | 0.0052 | 0.0026 | 83.8 | 8.2 | 1.3 | 7.1 | 50.7 | 26.6 |
| | | 0.85/0.15/2.0 | | | | | | | | | |
| Example 12 | p-X | Cu/Mn/Br | CuBr₂/MnBr₂ | 0.0052 | 0.0026 | 91.3 | 0.6 | 0.0 | 8.1 | 55.2 | 24.1 |
| | | 0.15/0.85/2.0 | | | | | | | | | |
| Example 13 | p-X | Co/Cu/Mn/Br | CoBr₂/CuBr₂/MnBr2 | 0.0052 | 0.0026 | 91.1 | 0.3 | 0.0 | 8.6 | 55.6 | 20.6 |
| | | 0.12/0.15/0.73/2.0 | | | | | | | | | |
| Example 14 | p-X | Ni/Cu/Br | NiBr₂/CuBr₂ | 0.0052 | 0.0026 | 89.7 | 0.7 | 0.0 | 9.5 | 59.0 | 20.3 |
| | | 0.85/0.15/2.0 | | | | | | | | | |
| Example 15 | p-X | Co/Cu/Fe/Br | Co(OAc)₂/CuBr₂/FeBr₃ | 0.0052 | 0.0031 | 92.9 | 0.0 | 0.0 | 7.1 | 61.0 | 24.0 |
| | | 0.75/0.083/0.167/2.0 | | | | | | | | | |
| Example 16 | p-X | Cu/Fe/Br | CuBr₂/FeBr₃ | 0.0052 | 0.0019 | 71.0 | 14.7 | 5.6 | 8.7 | 42.1 | 20.0 |
| | | 0.21/0.79/2.79 | | | | | | | | | |
| Example 17 | p-X | Co/Cu/Mn/Ni/Br | CoBr₂/CuBr₂/MnBr₂/NiBr₂ | 0.0052 | 0.0026 | 91.9 | 0.2 | 0.7 | 7.2 | 63.0 | 22.5 |
| | | 0.26/0.06/0.53/0.14/2.0 | | | | | | | | | |
| Example 18 | p-X | Co/Cu/Ni/Br | CoBr₂/CuBr₂/NiBr₂ | 0.0052 | 0.0026 | 92.3 | 0.3 | 0.6 | 6.9 | 65.2 | 22.9 |
| | | 0.58/0.14/0.28/2.0 | | | | | | | | | |
| Example 6 | p-X | Co/Cu/Br | CoBr₂/CuBr₂ | 0.0052 | 0.0026 | 92.0 | 0.0 | 0.5 | 7.6 | 66.4 | 21.9 |
| | | 0.9/0.1/2.0 | | | | | | | | | |

TABLE 4
Addition of hydrogen bromide to a cobalt/copper bromide catalyst and to a copper bromide catalyst.
Examples already described in earlier Tables are included for comparison purposes.

| | | Catalyst | | | | | Selectivity (%) | | | | Yield (%) | |
|---|---|---|---|---|---|---|---|---|---|---|---|---|
| Run | Feed | type | Catalyst | [Br], M | Br/Cu | [Cu], M | TA | p-Tol | 4-CBA | BA | TA | CO2 |
| Comp. Ex. 3 | p-X | H/Br | HBr | 0.0052 | | 0 | 30.8 | 52.2 | 7.1 | 7.2 | 8 | 29.8 |
| Example 1 | p-X | Cu/Br | CuBr₂ | 0.0052 | 2.0 | 0.0026 | 85.7 | 5.9 | 1.3 | 7.3 | 54.0 | 26.1 |
| Example 19 | p-X | Cu/Br | CuBr₂ | 0.00077 | 2.00 | 0.00039 | 21.6 | 58.4 | 10.2 | 7.2 | 12.2 | 17.6 |
| Example 20 | p-X | Cu/Na/Br | CuBr₂/NaBr | 0.0052 | 13.3 | 0.00039 | 49.6 | 33.7 | 5.8 | 9.3 | 32.6 | 18.1 |
| Example 21 | p-X | Cu/H/Br | CuBr₂/HBr | 0.0052 | 13.3 | 0.00039 | 90.8 | 1.4 | 0.2 | 7.6 | 53.9 | 20.4 |
| Example 22 | p-X | Cu/H/Br | CuBr₂/HBr | 0.0077 | 20.0 | 0.00039 | 94.5 | 0.0 | 0.0 | 5.5 | 55.9 | 20.5 |

TABLE 5
Effect of catalyst concentration to a copper bromide catalyst

| | | Catalyst | | | | Yield (%) | | | | |
|---|---|---|---|---|---|---|---|---|---|---|
| Run | Feed | type | Catalyst | [Br], M | [metals], M | p-Tol | 4-CBA | TA | BA | CO2 |
| Example 23 | p-X | Cu/H/Br | CuBr₂/HBr | 0.0062 | 0.0026 | 26.1 | 4.4 | 20.8 | 3.1 | 31.3 |
| Example 24 | p-X | Cu/H/Br | CuBr₂/HBr | 0.0124 | 0.0052 | 16.2 | 4.2 | 38.0 | 3.8 | 27.8 |
| Example 25 | p-X | Cu/H/Br | CuBr₂/HBr | 0.0247 | 0.0103 | 0.9 | 0.2 | 61.1 | 2.7 | 21.9 |
| Example 26 | p-X | Co/Cu/H/Br | CoBr₂/CuBr₂/HBr | 0.0062 | 0.0026 | 3.8 | 0.9 | 50.1 | 4.6 | 27.2 |
| Example 27 | p-X | Co/Cu/H/Br | CoBr₂/CuBr₂/HBr | 0.0124 | 0.0052 | 1.3 | 0.3 | 56.6 | 4.1 | 23.2 |
| Example 28 | p-X | Co/Cu/H/Br | CoBr₂/CuBr₂/HBr | 0.0247 | 0.0103 | 0.8 | 0.2 | 61.8 | 4.7 | 20.7 |

These experiments at 2% p-X.
Examples 26-28 Co:Cu 0.85:15

TABLE 6a
Near critical water oxidation of 4-methylanisole with a cobalt/copper bromide catalyst

| | | | | | | Yield (%) | | | | | |
|---|---|---|---|---|---|---|---|---|---|---|---|
| | | Catalyst | | | [metals], | 4-methyl | p-anis | p-anisic | p-hydroxy | p-hydroxy | |
| Run | Feed | type | Catalyst | [Br], M | M | anisole | aldehyde | acid | benzaldehyde | benzoic acid | CO2 |
| Example 29 | 4- | Co/Cu/Br | 0.9/0.1 | 0.0052 | 0.0026 | 6 | 4 | 49 | 5 | <1 | 14 |
| | methylanisole | | CoBr₂/CuBr₂ | | | | | | | | |

### Temperature 316-338° C.

TABLE 6b
Super critical water oxidation of o-xylene with a cobalt/copper bromide catalyst

| | | Catalyst | | | | Selectivity (%) | | | | | Yield (%) | |
|---|---|---|---|---|---|---|---|---|---|---|---|---|
| Run | Feed | type | Catalyst | [Br], M | [metals], M | OPA | Phth | oTolA | 2-CBA | BA | OPA | CO2 |
| Example 30 | o-xylene | Co/Cu/Br | 0.85/0.15 CoBr₂/CuBr₂ | 0.0103 | 0.0052 | 52.9 | 6.9 | 9.4 | 1.8 | 28.9 | 26.0 | 28.4 |

OPA = orthophthalic acid; Phth = Phthalide; oTolA = orthotoluic acid; 2-CBA = 2-carboxybenzaldehyde; BA = benzoic acid

1. An oxidation process for the production of an aromatic carboxylic acid, said process comprising contacting in the presence of a catalyst, within a continuous flow reactor, one or more precursor(s) of the aromatic carboxylic acid with an oxidant, such contact being effected with said precursor(s) and the oxidant in an aqueous solvent comprising water under supercritical conditions or near supercritical conditions, wherein said catalyst comprises copper.

2. A process according to claim 1 wherein the catalyst further comprises one or more additional metals other than copper.

3. A process according to claim 2 wherein said one or more additional metals are selected from transition metals.

4. A process according to claim 2 wherein said one or more additional metals are selected from manganese, cobalt, zirconium, hafnium, vanadium, chromium, molybdenum, iron, nickel and cerium.

5. A process according to claim 2 wherein the molar ratio [M]:[Cu] is no more than about 500:1 wherein [M] is the total molar amount of the other metal(s).

6. A process according to claim 2 wherein the catalyst further comprises cobalt.

7. A process according to claim 2 wherein the copper-containing catalyst comprises cobalt and the Co:Cu molar ratio is between about 1:1 and 10:1.

8. A process according to a claim 2 wherein the or each metal ion present in the catalyst is present as its bromide.

9. A process according to claim 1 wherein the catalyst comprises copper and cobalt, wherein at least one of said metals, is present as the bromide.

10. A process according to claim 1 further comprising the introduction of hydrogen bromide to the reaction mixture.

11. A process according to claim 10 wherein the amount of HBr is such that the molar ratio [HBr]:[M], where [M] is the total concentration of the metal ion(s) of the catalyst, is in the range of from about 1.0:1 to about 50.0:1.

12. A process according to claim 1 wherein said one or more precursors, oxidant and aqueous solvent constitute a single homogeneous phase in the reaction zone.

13. A process according to claim 1 wherein said contact of at least part of said precursor with said oxidant is contemporaneous with contact of said catalyst with at least part of said oxidant.

14. A process according to claim 1 wherein at least 98% wt of the aromatic carboxylic acid produced is maintained in solution during the reaction.

15. A process according to claim 1 wherein the aromatic carboxylic acid following reaction is precipitated from the reaction medium and contains no more than 5000 ppm by weight of aldehyde produced as an intermediate in the course of the reaction.

16. A process according to claim 1 wherein following the reaction the aromatic carboxylic acid-containing solution is processed to precipitate the aromatic carboxylic acid and the precipitate is separated from the mother liquor.

17. A process according to claim 1 wherein said aromatic carboxylic acid is selected from terephthalic acid, isophthalic acid, phthalic acid, trimellitic acid, naphthalene dicarboxylic acid, nicotinic acid and anisic acid.

18. A process according to claim 17 wherein said aromatic carboxylic acid is selected from terephthalic acid, isophthalic acid, phthalic acid and naphthalene dicarboxylic acid.

19. A process according to claim 17 wherein said aromatic carboxylic acid is terephthalic acid.

20. A process according to claim 1 wherein said precursor is selected from aromatic compounds having at least one substituent selected from alkyl, alcohol, alkoxyalkyl and aldehyde groups.

21. A process according to claim 1 wherein said precursor is selected from aromatic compounds having at least one substituent selected from alkyl groups.

22. A process according to claim 1 wherein said precursor is selected from aromatic compounds having at least one substituent selected from C₁₋₄ alkyl groups.

23. A process according to claim 19 wherein said precursor is para-xylene.

24. A process according to claim 1 wherein said aqueous solvent comprises water under near supercritical conditions in the liquid phase.

25. A process according to claim 1 wherein the operating temperature is in the range of from about 280 to about 480° C. and the operating pressure is in the range of from about 86 bara to about 350 bara.

26. A process according to claim 1 wherein the residence time for the reaction is no more than 10 minutes.

27. An aromatic carboxylic acid when produced by the process described in claim 1.
